(19) United States
(12) Patent Application Publication
(10) Pub. No.: US 2018/0177247 A1
(43) Pub. Date: Jun. 28, 2018

(54) COMBINATION OF URINE BAG AND SKIRT

(71) Applicant: Cheng Chieh Investment Co.,Ltd., Taoyuan City (TW)

(72) Inventor: Hsia-Sen LIN, Taoyuan City (TW)

(21) Appl. No.: 15/826,816

(22) Filed: Nov. 30, 2017

(30) Foreign Application Priority Data
Dec. 27, 2016 (TW) 105219763
Jul. 14, 2017 (TW) 106123592

(51) Int. Cl. A41D 1/14; A61F 5/453; A61F 5/455; A45C 13/10; B65D 85/18

(52) U.S. Cl. CPC A41D 1/14; A61F 5/453; A61F 5/4556; A41D 2300/332; B65D 85/18; A41D 2300/32; A41D 2300/322; A45C 13/1023

(57) ABSTRACT

A combination of urine bag and skirt includes a skirt having at least one opening for a user to stretch therein his/her hand(s) conveniently; a urine bag having a bag opening, and being provided inside of the skirt; and a urine guide having a guide opening and a guide joint such that the guide joint is sleeved to the bag opening. The user can wear the skirt as a shelter, and stretches his/her hand(s), through the at least one opening, into the skirt, where the user can hold, with his/her hand(s), the urine bag and the urine guide such that the guide joint is sleeved to the bag opening so as to urinate. Therefore, the combination of urine bag and skirt has merits on easy cleaning and replacement, and on urination sheltering.

### BACKGROUND OF THE INVENTION

### 1. Field of the Invention

The present invention relates to a combination of urine bag and skirt. The present invention comprises a skirt, a urine bag, and a urine guide, served to help users urinate and having merits on sheltering and easy cleaning.

### 2. Description of Related Art

It would be greatly troubled when one has to urinate but he/she cannot find a restroom, particularly for one in a long journey. This happens in the occasion of staying in the remote countryside where public toilets are not available, or traffic jams on the highway where urination is not convenient, causing uncomfortable for those making journeys, because they need to hold back urine. In the case of holding back urine for a long time, bladders will suffer a burden and worse, he/she may cause inflammation of bladders or of urethra and result in urinary system diseases, and as such, a great caution is necessary.

Further, in recent years, the problems of aging population and low fertility have been taken seriously, and thus health-caring industry develops accordingly, let alone social costs input to elders and patients increase year after year. For those receiving care, including medical care and home care, mostly less capable, limb atrophy, muscle weakness, hemiplegia, dementia, disability, long-term bed may be necessary, and because they normally cannot take care of themselves in daily life, assistance from others is a must.

To those receiving care, including patients and elders, considering their inconvenience resulted from postoperative healing or muscle aging, care givers can provide them a urine bag for use, so as to decrease the difficulties of urination or to avoid those receiving care from causing cleaning problems by excreting outside of a restroom. Therefore, the use of a urine bag is so important for those receiving care.

Further, to those receiving care, who require the use of a urine bag, when holding a urine bag during walking or laying the urine bag on the ground, may feel uncomfortable while bystanders are watching. Nor is it convenient for carrying the urine bag. As such, these problems have to be solved, and that a urine bag structure having merits on convenience and sheltering, plus easy replacement, becomes a choice for development.

Given the above, in designing a urine bag, lightweight and portable, dressing, body cleanliness, and easy to replace for users are the factors to be taken into account. Besides, sheltering for urination is also important to be considered. Development for such a urine bag having the merits is necessary so as to provide convenience for those unable to urinate for a long trip or move with difficulties.

### SUMMARY OF THE INVENTION

An object of the present invention is to provide a combination of urine bag and skirt, comprising a skirt having at least one opening for a user to stretch therein his/her hand(s) conveniently; a urine bag having a bag opening, and being provided in the inner of the skirt.

In an aspect of the invention, the combination further comprises a urine guide having a guide opening and a guide joint such that the guide joint is sleeved to the bag opening.

In an aspect of the present invention, the guide opening has a diameter greater than that of the guide joint.

In still another aspect of the present invention, the urine guide has a middle portion, where the middle portion has a diameter smaller than that of the guide joint, and the guide joint has a diameter greater than that of the bag opening.

In still another aspect of the present invention, at least one sealing element is provided at the bag opening.

In still another aspect of the present invention, the at least one sealing element relates to a zipper, a sealing clip, or a band.

In still another aspect of the present invention, the skirt includes a tightness control, where the tightness control serves to adjust the tightness of the skirt.

In still another aspect of the present invention, the tightness control may relate to an elastic band, a Velcro, a zipper, a strap, or a button.

In still another aspect of the present invention, the at least one opening of the skirt is provided at the front of the skirt, or at least one side of the skirt.

In still another aspect of the present invention, the urine bag is made of a material able to be cleaned and reusable, or of a disposable material.

In still another aspect of the present invention, the skirt and the urine bag can be packed in a carry bag.

Other objects, advantages, and novel features of the present invention will become more apparent from the following detailed descriptions in reference to the accompanying drawings.

### BRIEF DESCRIPTION OF THE DRAWINGS

FIG. 1 is a schematic view illustrating the skirt according to the example 1 of the present invention;

FIG. 2 is another schematic view illustrating the skirt according to the example 1 of the present invention;

FIG. 3 is a schematic view illustrating the state of use of the combination of urine bag and skirt according to the example 1 of the present invention;

FIG. 4 is a schematic view illustrating the urine bag according to the example 1 of the present invention;

FIG. 5 is a schematic view illustrating a carry bag packed inside with the combination of urine bag and skirt according to the present invention;

FIG. 6 is a schematic view illustrating the urine bag and the urine guide according to the example 2 of the present invention;

FIG. 7 is another schematic view illustrating the urine bag and the urine guide according to the example 2 of the present invention;

FIG. 8 is a schematic view illustrating the state of use of the combination of urine bag and skirt according to the example 2 of the present invention; and

FIG. 9 is a schematic view illustrating the urine bag of the combination of urine bag and skirt, being sealed after use, according to the example 2 of the present invention.

### DETAILED DESCRIPTION OF THE PREFERRED EMBODIMENTS

Innovative technical features, including specific features, according to the present invention, are disclosed in the claims, wherein the technical features are described in detail as follows, accompanied with the specification and drawings, under a preferable comprehension and principle of implementation of the present invention.

Terms regarding technology and science, as described in the specification and the claims, except otherwise defined, all fall within the definitions known to those of common knowledge in the art. Terms in singular form such as “a,” “one,” “the,” or their approximate terms, except otherwise indicated, all refer to more than one object; and that the terms “or,” “as well,” “and,” except otherwise indicated, all refer to “or/and.” Further, both the terms “comprising” and “including” refer to open-type conjunctions without limitation thereto. These explanations for definition merely refer to a definition to terminologies, rather than a limitation to the subject of the invention, and unless otherwise indicated, materials referred to in the present invention can be available in the market.

### Example 1

The present invention provides a combination of urine bag and skirt, comprising a skirt 1 and at least one urine bag 2.

According to the present invention, the skirt may refer to a long skirt, a short skirt, a narrow skirt, or an apron, and that pattern and length of the skirt are not the technical features referred to in the present invention.

According to the present invention, the urine bag is foldable and is disposed in the inner of the skirt 1.

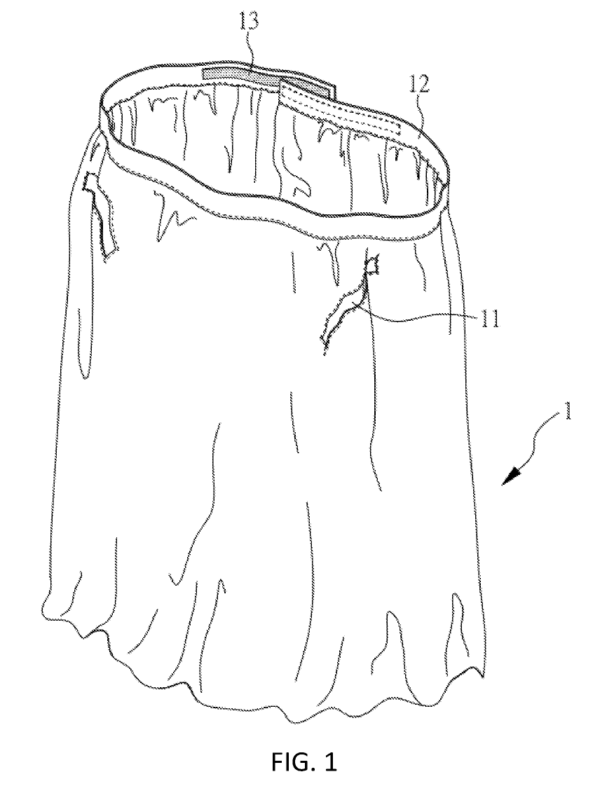

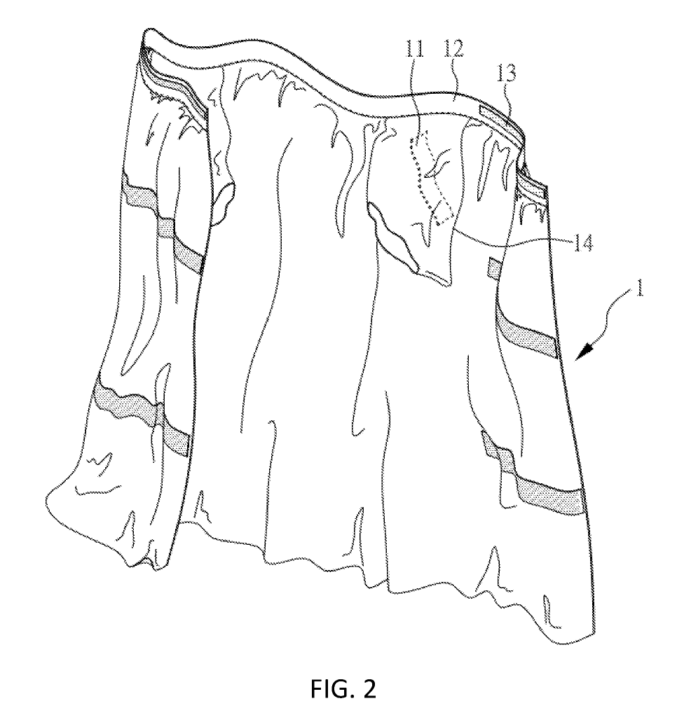

Referring to FIGS. 1 and 2, according to the present invention, the skirt 1 includes a waistband 12 and a tightness control 13, where the tightness control 13 is arranged on the waistband 12, such that the waistband 12 can be formed as an annular structure and serve to adjust the tightness of the skirt 1, so as to facilitate wearing and taking off the skirt 1. The tightness control 13 may relate, but not limited, to an elastic band, a Velcro, a zipper, a button, or a strap.

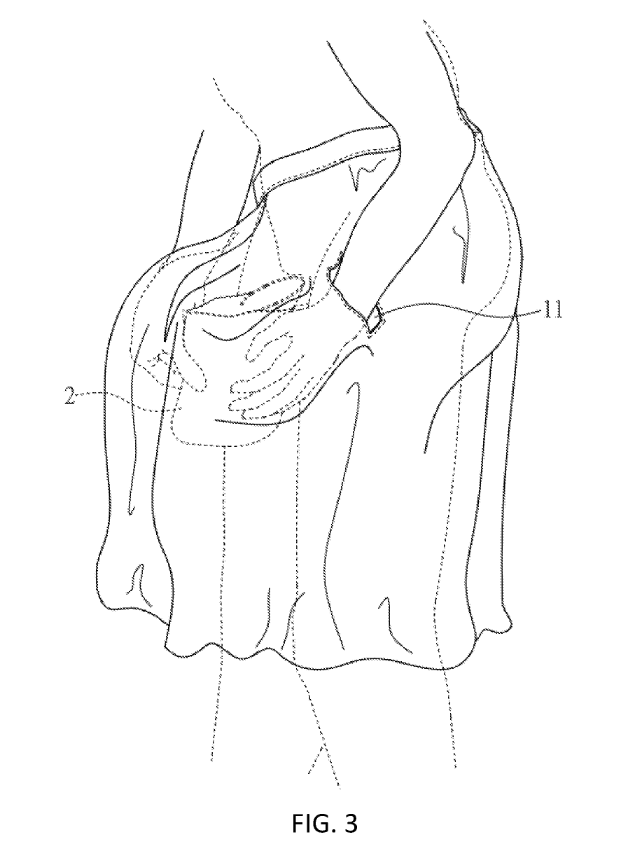

Further referring to FIG. 3, according to the present embodiment, the skirt 1 includes at least one opening 11, and preferably two openings 11. When the user wears the skirt 1, he/she puts his/her hands into the skirt 1 via the openings 11, and then holds the urine bag 2 with the hands.

Preferably, the openings 11 are provided at the front of the skirt 1, or at the sides of the skirt 1. The so-called “at the front” refers to a place extending downward from the abdomen of a user; and “at the sides” refers to when the user stands and he/she hangs down hands at his/her sides. Preferably, the opening 11 is connected to an inner bag 14, wherein the inner bag 14 is a bag-shaped structure which extends from the opening 11 to the inner of the skirt 1. The difference between the inner bag 14 and a normal pocket is that the inner bag 14 has an opening which allows the user of the present invention to stretch hands into the skirt via the inner bag 14, holding the urine bag 2 with hands, and urinating. As demonstrated in FIG. 3, the present invention has merits both on convenient manipulation and urination sheltering.

Moreover, in another embodiment of the invention, the skirt 1 selectively comprises at least one fixing unit 15 disposed in the inner of the skirt 1, which is used to hang or place the urine bag 2. The said inner of the skirt 1 refers to the side facing to the user's body while wearing the skirt 1. Preferably, the fixing unit includes, but not limited to, hooks or clamps.

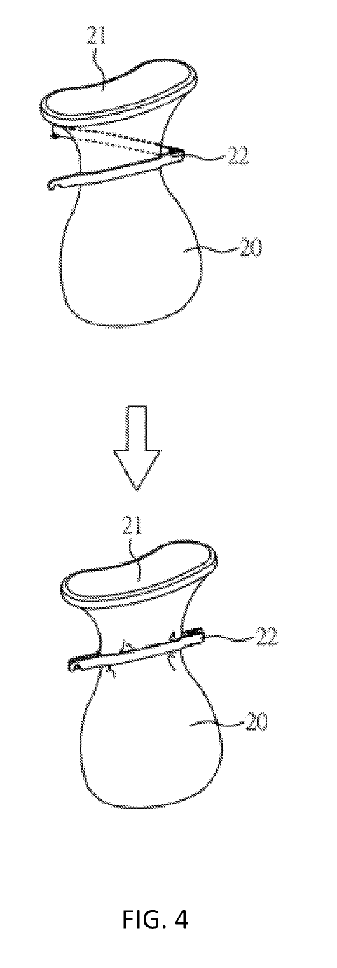

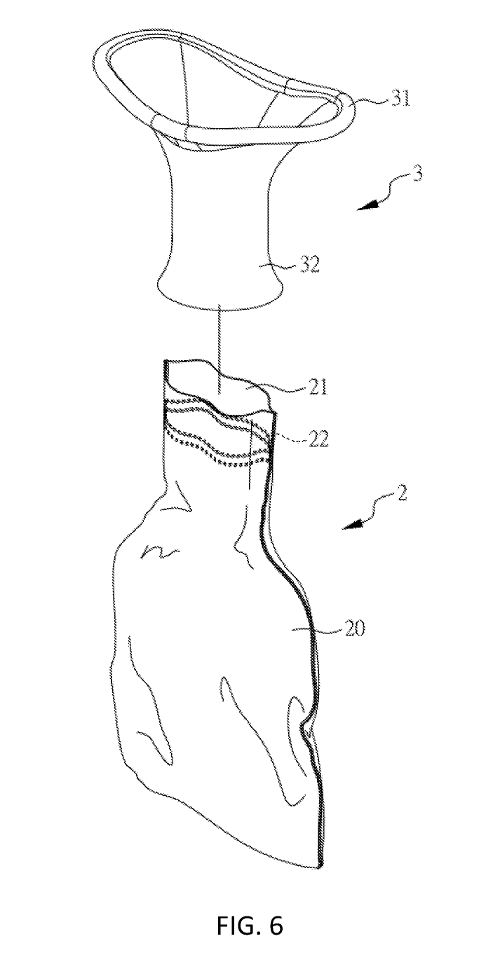

Further referring to FIG. 4, the urine bag 2 is made of soft material with elasticity so as to facilitate containing urine. The urine bag 2 includes a bag opening 21 and a bag body 20. When the user is using the urine bag 2, the urine is discharged into the bag opening 21 and kept in the bag body 20. The bag opening 21 may have an appearance like an ergonomic arc opening, and further, a sealing element 22 is provided for sealing or binding the bag opening 21 so as to avoid leakage of urine. The sealing element 22 may be arranged on the bag body 20, or alternatively, may be an individual element which is not arranged on the bag body 20. The sealing element 22 may relate, but not limited, to a zipper, a sealing clip, or a band, which may be replaced with other element having a sealing function (illustrated as a sealing clip in FIG. 4 and as zippers in FIG. 6). Preferably, the sealing element relates to one or two zippers (FIG. 6). Preferably, the sealing element 22 comprises a hang ring which is used to attach to a fixing unit 15 of the skirt to fix the urine bag 2 on the skirt 1.

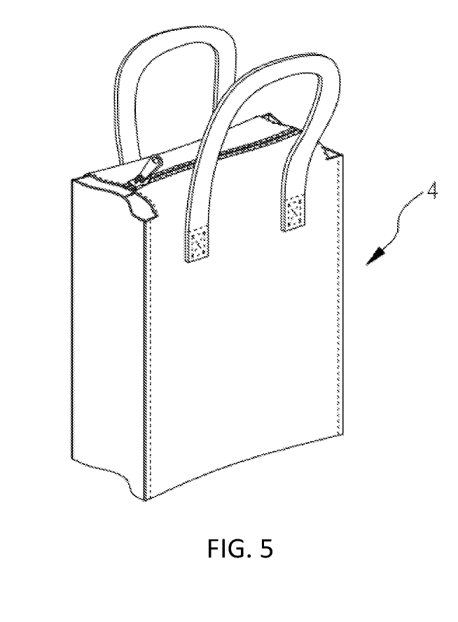

Further, according to the present invention, the combination of urine bag and skirt can be packed in a carry hag 4, by receiving therein the skirt 1 and the urine bag 2, so as to accommodate and carry the same (see FIG. 5).

Still further, the material of the combination of urine bag and skirt, according to the present invention, can be made of, but not limited to, polyester fiber or rayon.

### Example 2

The present invention provides a combination of urine bag and skirt, comprising a skirt 1, a urine bag 2, and a urine guide 3. The present example is similar to the example 1, and the feature of example 1 can be applied to the example 2, unless defined otherwise.

Referring to FIG. 1, according to the present invention, the skirt 1 includes a waistband 12 and a tightness control 13, where the tightness control 13 is arranged on the waistband 12, such that the waistband 12 can be formed as an annular structure and serve to adjust the tightness of the skirt 1, so as to facilitate wearing and taking off the skirt 1. The tightness control 13 may relate, but not limited, to an elastic band, a Velcro, a zipper, a button, or a strap.

Further, according to the present embodiment, the skirt 1 includes at least one opening 11, and preferably two openings 11. Preferably, the openings 11 are provided at the front of the skirt 1, or at the sides of the skirt 1. The so-called “at the front” refers to a place extending downward from the abdomen of a user; and “at the sides” refers to when the user stands and he/she hangs down hands at his/her sides. When the user wears the skirt 1, he/she stretches his/her hands into the skirt 1 via the openings 11, and then holds the urine bag 2 with the hands.

Referring to FIG. 6, the urine bag 2 is made of soft material with elasticity so as to facilitate containing urine. The urine bag 2 includes a bag opening 21 and a bag body 20. When the user is using the urine bag 2, the urine is discharged into the bag opening 21 and kept in the bag body 20. The bag opening 21 may have an appearance like an ergonomic arc opening, and further, a sealing element 22 is provided for sealing or binding the bag opening 21 so as to avoid leakage of urine. The sealing element 22 may be arranged on the bag body 20, or alternatively; may be an individual element which is not arranged on the bag body 20. The sealing element 22 may relate, but not limited, to a zipper, a sealing clip, or a band, which may be replaced with other element having a sealing function (illustrated as zippers in FIG. 6). Preferably, Preferably, the sealing element relates to one or two zippers. Additionally, the bag body can forms a body edge by hot pressing while manufacturing the bag body 33 so as to reinforce and strengthen the bag body 33, as disclosed in FIG. 6 that the bag body has a hot-pressing body edge.

According to the present embodiment, the urine guide 3 is provided for introducing the urine into the urine bag 2, where the urine guide 3 includes a guide opening 31 and a guide joint 32. The guide joint 32 is sleeved to the bag opening 21 of the urine bag 2. Preferably, the urine guide 3 has a funnel-like shape, where the guide opening 31 has a diameter greater than that of the guide joint 32. Further, preferably, the urine guide 2 has a thickness at the guide opening 31 greater than a thickness at the guide joint 32.

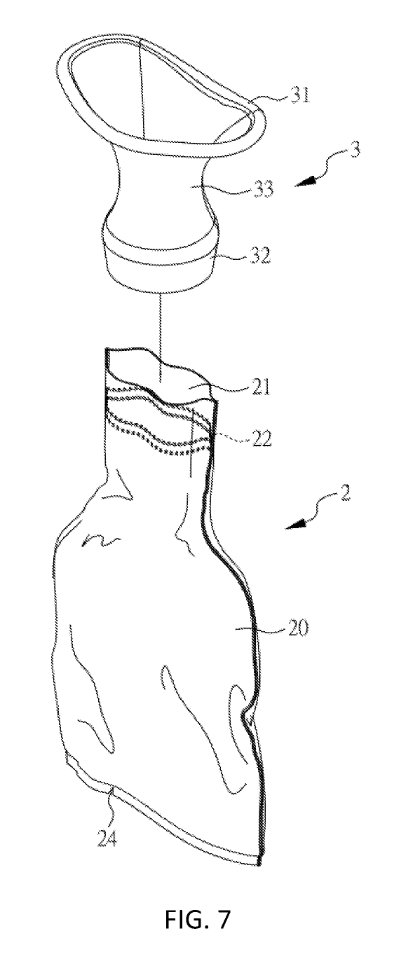

In another embodiment, as shown in FIG. 7, the urine guide 3 has a funnel-like shape and a middle portion 33, wherein the middle portion 33 has a diameter smaller than that of a guide joint 32. Besides, the guide joint 32 has a diameter greater than that of the bag opening 21 of the urine bag 2. In view of the fact that the urine bag 2 is made of materials of elasticity, when the guide joint 32 is sleeved to the bag opening 21 and is positioned in the middle portion 23, the urine bag 2 will not fall off easily. Further, in another embodiment, a bag body 23 of the urine bag 2 may further include a tear opening 24 which can be easily torn open, such that after urination, the user can open the urine bag 2 from the tearing opening 24 so as to pour out the urine.

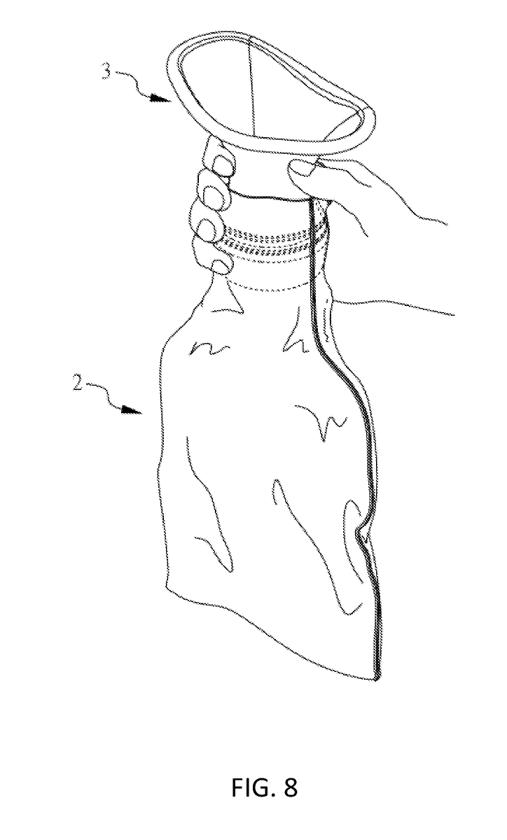

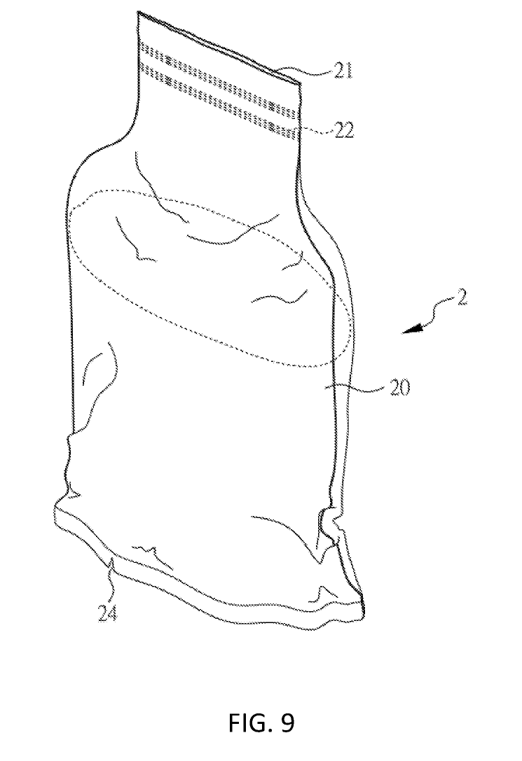

According to the present embodiment, the combination of urine bag and skirt comprises the skirt 1, the urine bag 2, and the urine guide 3. The user can wear the skirt 1 as a shelter, and stretches his/her hands, through the openings 11, into the skirt 1, where the user can hold, with hands, the urine bag 2 and the urine guide 3 such that the guide joint 32 of the urine guide 3 is sleeved to the bag opening 21 of the urine bag 2 so as to urinate (see FIG. 8). Thereafter, the user holds the urine bag 2 at the bag opening 21, and separates the urine guide 3 from the urine bag 2, and then seals the bag opening 21 of the urine bag 2 with the sealing element 22 (e.g. dual zippers), where the urine bag 2 can be disposable or reusable (see FIG. 9). Therefore, it is appreciated that the combination of urine bag and skirt, according to the present embodiment, has merits both on convenient manipulation and urination sheltering.

Although the present invention has been explained in relation to its preferred embodiments, it is to be understood that the embodiments, given above, are proposed for exemplification rather than for limitation, and that the present invention can be implemented, through many other possible modifications and variations in line with the features of the present invention, by those skilled in the art. The scope of the present invention, as hereinafter claimed, covers the method and structure previously described, and the equivalents therewith.

What is claimed is:

1. A combination of urine bag and skirt, comprising:
a skirt, having at least one opening for a user to stretch therein his/her hand(s) conveniently; and
a urine bag, having a bag opening, and being provided in the inner of the skirt.

2. The combination of urine bag and skirt as claimed in claim 1, wherein the combination further comprising a urine guide, having a guide opening and a guide joint such that the guide joint is sleeved to the bag opening.

3. The combination of urine bag and skirt as claimed in claim 2, wherein the guide opening has a diameter greater than that of the guide joint.

4. The combination of urine bag and skirt as claimed in claim 2, wherein the urine guide has a middle portion, the middle portion has a diameter smaller than that of the guide joint, and the guide joint has a diameter greater than that of the bag opening.

5. The combination of urine bag and skirt as claimed in claim 1, wherein at least one sealing element is provided at the bag opening.

6. The combination of urine bag and skirt as claimed in claim 5, wherein the at least one sealing element relates to a zipper, a sealing clip, or a band.

7. The combination of urine bag and skirt as claimed in claim 1, wherein the skirt includes a tightness control served to adjust the tightness of the skirt.

8. The combination of urine bag and skirt as claimed in claim 6, wherein the tightness control relates to an elastic band, a Velcro, a zipper, a strap, or a button.

9. The combination of urine bag and skirt as claimed in claim 1, wherein the urine bag is made of a material able to be cleaned and reusable, or of a disposable material.

10. The combination of urine bag and skirt as claimed in claim 1, wherein the skirt and the urine bag can be packed in a carry bag.
